(19) United States
(12) Patent Application Publication
(10) Pub. No.: US 2018/0153458 A1
(43) Pub. Date: Jun. 7, 2018

(54) STRESS FEEDBACK FOR PRESENTATIONS

(71) Applicant: MICROSOFT TECHNOLOGY LICENSING, LLC, Redmond, WA (US)

(72) Inventor: Patrick Desjardins, Redmond, WA (US)

(73) Assignee: MICROSOFT TECHNOLOGY LICENSING, LLC, Redmond, WA (US)

(21) Appl. No.: 15/371,616

(22) Filed: Dec. 7, 2016

(51) Int. Cl. A61B 5/16; H04L 29/08; G06T 11/20; A61B 5/0205; A61B 5/00; A61B 5/01

(52) U.S. Cl. CPC A61B 5/165; H04L 67/02; G06T 11/206; A61B 5/02055; A61B 5/024; A61B 5/01; A61B 5/7275; A61B 5/742; A61B 5/443

(57) ABSTRACT

Presentations are a beneficial tool for sharing information between a presenter and audience. However, because presentations can be nerve-wracking and stressful for the presenter, the information may not always be communicated in the most clear and efficient manner. Thus, embodiments are directed to providing stress feedback for presentations so that the presenter may be able to learn and improve for the next presentation. For example signals associated with body regulation of a presenter may be received throughout the presentation from one or more sensors affixed to a body of the presenter, and time information may be received from a productivity service as the presentation is progressed by the presenter. A stress level of the presenter may be determined based on the received signals, the determined stress level of the presenter may be correlated with the time information, and the correlation may be provided far display.

### BACKGROUND

Presentations are a beneficial tool for sharing information between a presenter and target audience. Often, following the presentation, presenters want to know how well they did in communicating that information and how they can improve. Because presentations can be nerve-wrecking and stressful for the presenter, the information to be shared may not always be communicated in the most clear and efficient manner. However, if feedback associated with the presenter's stress levels during the presentation could be provided to show the areas of the presentation that made the presenter more nervous, the presenter may be able to learn and improve for the next presentation.

### SUMMARY

This summary is provided to introduce a selection of concepts in a simplified form that are further described below in the Detailed Description. This summary is not intended to exclusively identify key features or essential features of the claimed subject matter, not is it intended as an aid in determining the scope of the claimed subject matter.

Embodiments are directed to provision of stress feedback for a presentation. Signals associated with body regulation of a presenter may be received throughout the presentation from one or more sensors affixed to a both of the presenter. Additionally, as the presentation is progressed by the presenter time information may be received from a productivity service configured to execute the presentation. A stress level of the presenter may be determined based on the received signals, the determined stress level of the presenter may be correlated with the time information, and the correlation may be provided for display.

These and other features and advantages will be apparent from a reading of the following detailed description and a review of the associated drawings. It is to be understood that both the foregoing general description and the following detailed description are explanatory and do not restrict aspects as claimed.

### BRIEF DESCRIPTION OF THE DRAWINGS

FIG. 1 includes a display diagram illustrating an example network environment where a system to provide stress feedback for a presentation may be implemented;

FIG. 2 includes a display diagram illustrating another example network environment where a system to provide stress feedback for a presentation may be implemented;

FIG. 3 includes a display diagram illustrating a further example network environment where a system, to provide stress feedback for a presentation may be implemented;

FIG. 4 includes a display diagram illustrating an example scheme to provide stress feedback for a presentation;

FIGS. 5A-B include display diagrams illustrating example correlations provided for display;

FIG. 6 is a networked environment, where a system according to embodiments may be implemented;

FIG. 7 is a block diagram of an example general purpose computing device, which may be used to provide stress feedback for a presentation; and

FIG. 8 illustrates a logic flow diagram of a method to provide stress feedback for a presentation, arranged in accordance with at least some embodiments described herein.

### DETAILED DESCRIPTION

As briefly described above, embodiments are directed to provision of stress feedback for a presentation. One or more sensors may be affixed to a body of the presenter to collect signals associated with body regulation of the presenter throughout the presentation, such as heart rate, body temperature, and galvanic skin response. A productivity service may be configured to execute the presentation through a web or client application and collect time and location information as the presentation is progressed by the presenter. A hosted service communicatively coupled to the sensors and the productivity service may be configured to receive the signals from the sensors and the time and location information from the productivity service throughout the presentation. The hosted service may then be configured to determine a stress level of the presenter based on the received signals, correlate the determined stress level of the presenter with the time and location information, and provide the correlation for display. The correlation may be displayed in real-time as the presenter is giving the presentation and/or may be stored in a database associated with the hosted service such that the correlation may be retrieved at a later time for analysis and comparison with one or more other correlations associated with the presentation.

In the following detailed description, references are made to the accompanying drawings that form a part hereof, and in which are shown by way of illustrations, specific embodiments, or examples. These aspects may be combined, other aspects may be utilized, and structural changes may be made without departing from the spirit or scope of the present disclosure. The following detailed description is therefore not to be taken in a limiting sense, and the scope of the present invention is defined by the appended claims and their equivalents,

While some embodiments will be described in the general context of program modules that execute in conjunction with an application program that runs on an operating system on, a personal computer, those skilled in the art will recognize that aspects may also be implemented in combination with other program modules.

Generally, program modules include routines, programs, components, data structures, and other types of structures that perform particular tasks or implement particular abstract data types. Moreover, those skilled in the art will appreciate that embodiments may be practiced with other computer system configurations, including hand-held devices, multiprocessor systems, microprocessor-based or programmable consumer electronics, minicomputers, mainframe computers, and comparable computing devices. Embodiments may also be practiced in distributed computing environments where tasks are performed by remote processing devices that are linked through a communications network. In a distributed computing environment, program modules may be located in both local and remote memory storage devices.

Some embodiments may be implemented as a computer-implemented process (method), a computing system, or as an article of manufacture, such as a computer program product or computer readable media. The computer program product may be a computer storage medium readable by a computer system and encoding a computer program that comprises instructions for causing a computer or computing system to perform example process(es). The computer-readable storage medium is a computer-readable memory device. The computer-readable storage medium can for example be implemented via one or more of a volatile computer memory, a non-volatile memory, a hard drive, a flash drive, a floppy disk, or a compact disk, and comparable hardware media.

Throughout this specification the term “platform” may be a combination of software and hardware components for providing stress feedback for a presentation. Examples of platforms include, but are not limited to, a hosted service executed over a plurality of servers, an application executed on a single computing device, and comparable systems. The term “server” generally refers to a computing device executing one or more software programs typically in a networked environment. However, a server may also be implemented as a virtual server (software programs) executed on one or more computing devices viewed as a server on the network. More detail on these technologies and example operations is provided below.

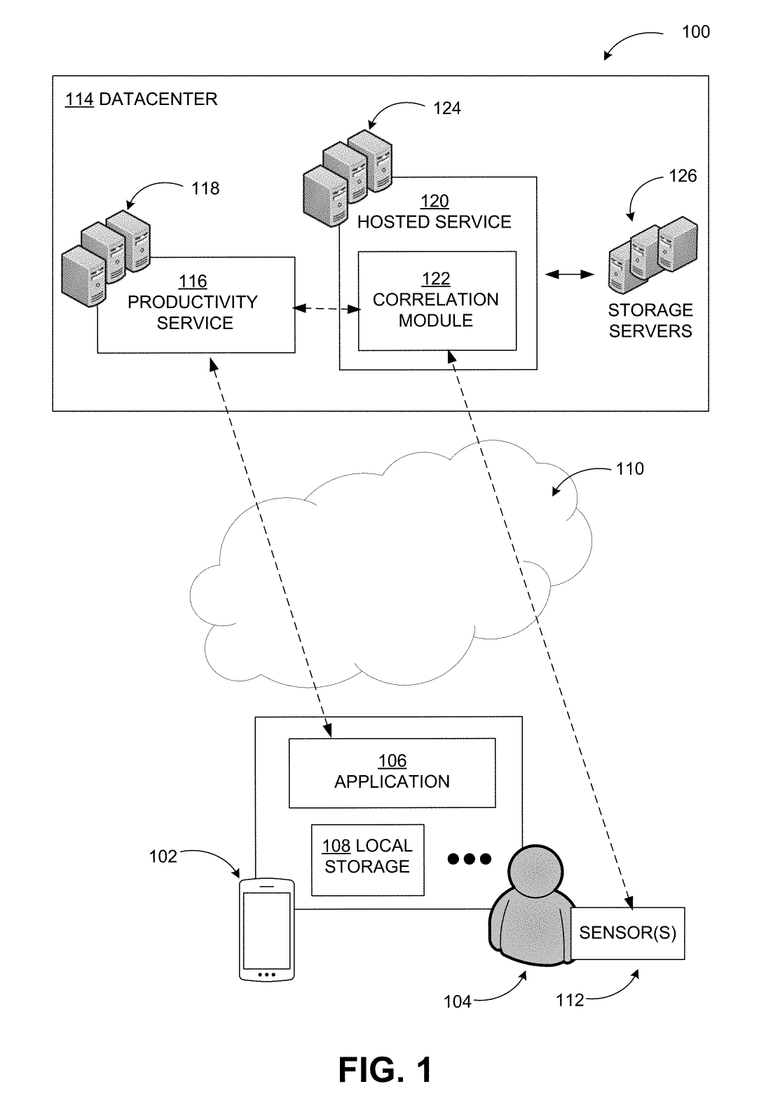

FIG. 1 includes a display diagram illustrating an example network environment where a system to provide stress feedback for a presentation may be implemented.

As illustrated in diagram 100, an example system may include a datacenter 114 hosting a productivity service 116 configured to, among other things execute a presentation and a hosted service 120 configured to provide stress feedback for a presentation. In other example systems, the productivity service 116 may be hosted by another datacenter. The datacenter 114 may include one or more processing servers 118 configured to execute the productivity service 116 and one or more additional processing servers 124 configured to execute the hosted service 120, among other components. In some embodiments, at least one of the additional process ng servers 1124 may be operable to execute a correlation module 122 of the hosted service 120, where the correlation module 12 may be integrated with the hosted service 120 to provide stress feedback for a presentation. Stress feedback may allow a presenter 104 to analyze how the presentation went and how to improve the presentation the future. In other embodiments, this function may be inherently present within the productivity service 116 itself. The datacenter 114 may also include one or more storage servers 126 configured to manage one or more data stores comprising data collected or stored by the productivity service 116 and/or data associated with a correlation produced by the correlation module 122. As described herein, be productivity service 116, the hosted service 120 and/or correlation module 122 may be implemented as software, hardware, or combinations thereof.

In some embodiments, the productivity 116 may be configured to interoperate with various applications to enable the presenter 104 to create and display a presentation. For example, as illustrated in the diagram 100, the presenter 104 may execute a thin (e.g., a web browser) or a thick (e.g., a locally installed client application) version of an application 106 through a device 102 with which the productivity service 116 may be configured to integrate and interoperate with over one or more networks, such as network 110. The application 106 may be a word-processing application, a presentation application, a notebook application, or a spreadsheet application that is capable of displaying a continuous presentation based on individual slides, pages, or other similar segments, for example. The presentation may be, stored locally, at local storage 108 of the device 102 or remotely at the storage servers 126 of the datacenter 114. The device 102 may include a desktop computer, a laptop computer, a tablet computer, a vehicle mount computer, a smart phone, or a wearable computing device, among other similar devices. The presenter 104 may also have one or more sensors 112 affixed to his or her body. The sensors 112 may be remote sensors or may be integrated within a wearable computing device, such as a watch or a band. The hosted service 120 may be communicatively coupled to both the productivity service 116 and one or more sensors 112 affixed to a body of the presenter 104, where a communication interface may facilitate communication between the productivity service 116, the sensors 112, and the hosted service 120 over the network 110.

In an example embodiment, the productivity service 116 may enable display of the presentation. As the presentation is progressed by the presenter 104, the productivity service 116 may be configured to collect time and/or location information. The time may begin at the start of the presentation and complete at the end of the presentation. The location information may include a slide number, a page number, paragraph number, a line number, or other similar segmentation indicator. Additionally, the sensors 112 may be configured to collect signals associated with body regulation of the presenter 104 throughout the presentation. The collected signals may include heart rate, body temperature, and galvanic skin response, for example, along with signals associated with any other body regulations indicative of stress and detectable via body contact based sensors.

The correlation module 122 may be configured to receive the signals from the sensors 112 and the time and/or location information from the productivity service 116 throughout the duration of the presentation. The correlation module 122 may then be configured to determine a stress level of the presenter 104 based on the received signals, correlate the determined stress level of the presenter 104 with the time and location information, and provide the correlation for display. In some examples, the correlation may be displayed as a graph, where the x-axis represents time and/or location information and the y-axis represents the stress level of the presenter.

The correlation module 122 may be configured to display the correlation in real-time as the presenter 104 is giving the presentation. Additionally and/or alternatively, the correlation module 122 may be configured to store the correlation within the storage servers 126 such that the correlation may be retrieved at a later time for analysis and'or comparison, For example, after the presentation, the presenter 104 may want to know how well he or she did and how they can improve in the future. Therefore, the correlation can be retrieved from the storage servers 126 and may be analyzed to show at which slides, pages, paragraphs, lines, and/or segments of the presentation, the presenter 104 was the most stressed. Additionally, the presenter 104 may give the presentation on multiple occasions. A correlation may be provided on each occasion and stored in the storage servers 126. The correlations may then be retrieved from the storage servers 126, analyzed, and compared in order to see if the presenter 104 has improved. In another example, other presenters, such as team, group, or organizational members affiliated with the presenter 104 may give a same presentation. Correlations associated with the other presenters may also be captured and stored such that feedback may be provided to the team, group, or organization as a whole.

In some embodiments, one or more additional sensors may be affixed to bodies of one or more audience members listening to the presentation. The additional sensors may be configured to collect signals associated with body regulation of the audience members throughout the presentation. The signals collected may be similar to those collected for the presenter 104. The hosted service 120 may be communicatively coupled to the additional sensors through the communication interface over the network 110, and the correlation module 122 may be configured to receive the signals from the additional sensors. The correlation module may then be configured to determine a stress level of the audience members based on the received signals, correlate the determined stress level of the audience members with the time and location information, and provide the correlation associated with the audience members for display. In some examples, the correlation associated with the presenter 104 and the correlation associated with the audience members may be aggregated for display.

As previously discussed, presentations a beneficial tool for sharing information between a presenter and target audience. However, because presentations can be nerve-wracking and stressful for the presenter, the information to be shared may not always be communicated in the most clear and efficient manner. Therefore, embodiments as described herein are directed to viding feedback associated with the presenter's stress levels during the presentation to show the presenter areas of the presentation at made him or her more nervous or stressed, allowing the presenter to learn and improve for the next presentation. The correlation of stress level to time and location information may be displayed real-time as a user-friendly graph, improving usability and increasing user interactivity. Additionally, providing analysis and comparison tools for correlations representing multiple occurrences of the presentation by the same presenter or correlations representing a same presentation given by multiple presenters from a same team, group, or organization may further improve usability and increase user interactivity.

Embodiments, as described herein, address a need that arises from very large scale operations created by software-based services that cannot be managed by humans. The actions/operations described herein are not a mere use of a computer, but address results o z system that is a direct consequence of software used as a service offered in conjunction with large numbers of devices and users sharing information through presentation.

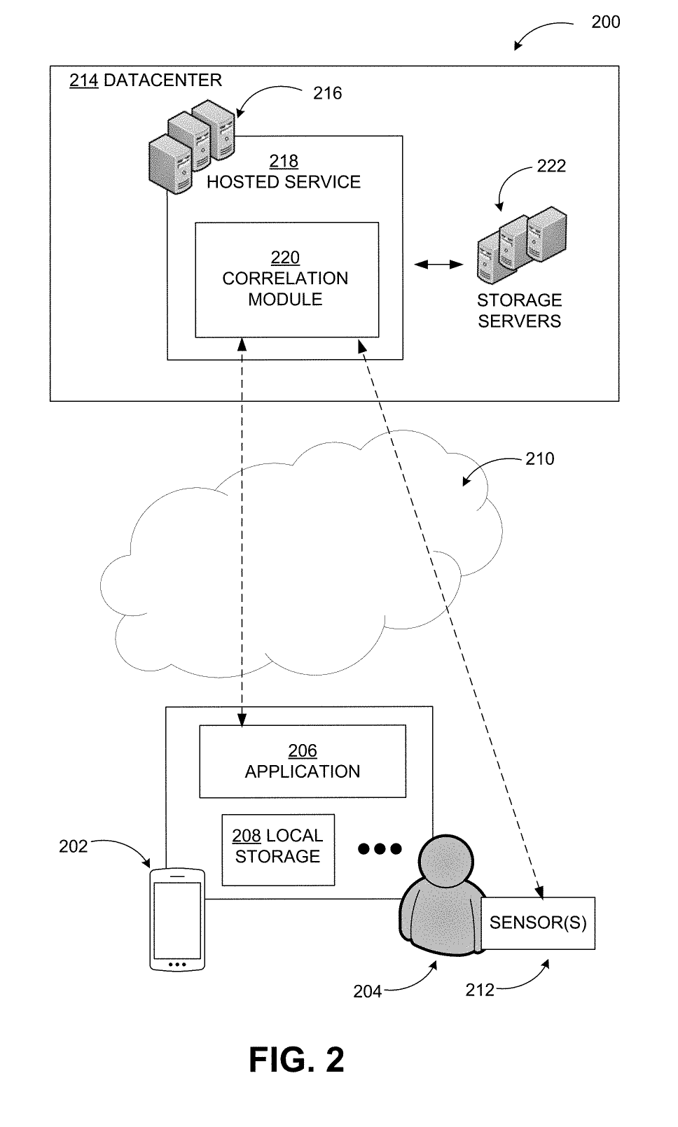

FIG. 2 includes a display diagram illustrating another example network environment where a system to provide stress feedback for a presentation may be implemented.

As illustrated in diagram 200, an example system may include a datacenter 214 hosting a service 218 configured to provide stress feedback for a presentation. The datacenter 214 may include one or more processing servers 216 configured to execute the hosted service. 218, among other components. In some embodiments, at least one of the processing servers 216 may be operable to execute a correlation module 220 of the hosted service 218, where the correlation module 220 may be integrated with the hosted service 218 to provide stress feedback for a presentation to allow a presenter 204 to analyze how the presentation went and how to improve the presentation in the future. The datacenter 214 may also is include one or more storage servers 222 configured to manage one or more data stores comprising data associated with a correlation produced by the correlation module 220. As described herein, the hosted service 218 and/or correlation module 220 may be implemented as software, hardware, or combinations thereof.

In some embodiments, the hosted service 218 may be configured to intemperate with various applications. For example, as illustrated in the diagram 200, the presenter 204 may execute a a thick (e.g., a locally installed client application) version of an application 206 through a device 202 with which the hosted service 218 may be configured to communicate with over one or more networks, such as network 210. The application 206 may be a word-processing application, a presentation application, a notebook application, or a spreadsheet application that is capable of displaying a continuous presentation based on individual slides, pages, or other similar segments, for example. The presentation may be stored locally at local storage 208 of the device 202. The device 202 may include a desktop computer, a laptop computer, a tablet computer, a vehicle mount computer, a smart phone, or a wearable computing device, among other similar devices. The presenter 204 may also have one or more sensors 212 affixed to his or her body. The sensors 212 may be remote sensors or may be integrated within a wearable computing device, such as a watch or a band.

In an example embodiment, the application 206 may execute the presentation, and collect time and/or location information as the presentation is progressed by the presenter 204. The correlation module 220 may be configured to receive signals associated with body regulation of the presenter 204 throughout the presentation from the sensors 212. The correlation module 220 may also be configured to receive the collection time and/or location information from the application 106. The correlation module 220 may then determine a stress level of the presenter 204 based on the received signals, correlate the determined stress level of the presenter 204 with the time and/or location information collected, and provide the correlation for display.

The correlation module 220 may be configured to display the correlation in real-time as the presenter is giving the presentation through a website associated with the hosted service 218, for example, or provide the correlation for display through the application 206. Additionally and/or alternatively, the correlation module 220 may be configured to store the correlation in the storage servers 222 such that the correlation may be retrieved at a later time for analysis and/or comparison.

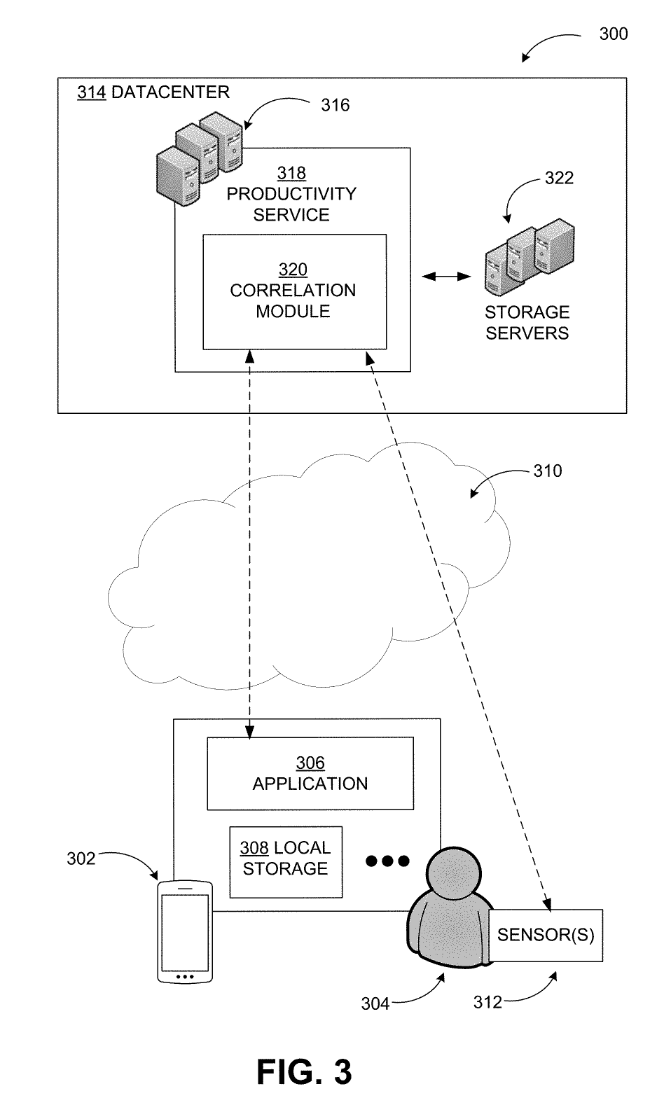

FIG. 3 includes a display diagram illustrating a further example network environment where a system to provide stress feedback for a presentation may be implemented.

As illustrated in diagram 300, an example system may include a datacenter 314 hosting a productivity service 318 configured to execute a presentation and provide stress feedback for the presentation. The datacenter 314 may include one or more processing servers 316 configured to execute the productivity service 318, among other components. In some embodiments, at least one of the processing servers 316 may be operable to execute a correlation module 320 of the productivity service 318, where the correlation module 320 may be integrated with the productivity service 318 to provide the stress feedback for the presentation that allows a presenter 304 to analyze how the presentation went and how to improve the presentation in the future. The datacenter 314 may also include one or more storage servers 322 configured to manage one or more data stores comprising data associated with a correlation produced by the correlation module 320. As described herein, the productivity service 318 and/or correlation module 220 may be implemented as software, hardware, or combinations thereof.

In some embodiments, the productivity service 318 may be configured to interoperate with various applications to enable the presenter 304 to create and display a presentation. For example, as illustrated in the diagram 300, the presenter 304 may execute a thin (e.g., a web browser) or a thick (e.g., a locally installed client application) version of an application 306 through a device 302 with which the productivity service 318 may be configured to integrate and interoperate with over one or more networks, such as network 310. The application 306 may be a word-processing application, a presentation application, a notebook application, or a spreadsheet application that is capable of displaying a continuous presentation based on individual slides, pages, or other similar segments, for example. The presentation may be stored locally at local storage 308 of the device or remotely at the storage servers 322 of the datacenter 314. The device 302 may include a desktop computer, a laptop computer, a tablet computer, a vehicle mount computer, a smart phone, or a wearable computing device, among other similar devices. The presenter 304 may also have one or more sensors 312 affixed to his or her body. The sensors 312 may be remote sensors or may be integrated within a wearable computing device, such as a watch or a band.

In an example embodiment, the productivity service 318 may enable display of the presentation through the application 306. As the presentation is progressed by the presenter 304. the productivity service 318 may be configured to collect time and/or location information. The time may begin at the start of the presentation and complete at the end of the presentation. The location information may include a slide number, a page number, paragraph number, a line number, or other similar segmentation indicator. The correlation module 320 may be configured to receive signals associated with body regulation of the presenter 304 throughout the presentation from the sensors 312, determine a stress level of the presenter 304 based on the received signals, correlate the determined stress level of the presenter 104 with the time and/or location information collected, and provide the correlation for display.

The correlation module 320 may be configured to display the correlation in real-time as the presenter 304 is giving the presentation through the application 306. Additionally and/or alternatively, the correlation module 320 may be configured to store the correlation in the storage servers 322 such that the correlation may be retrieved at a later time for analysis and/or comparison.

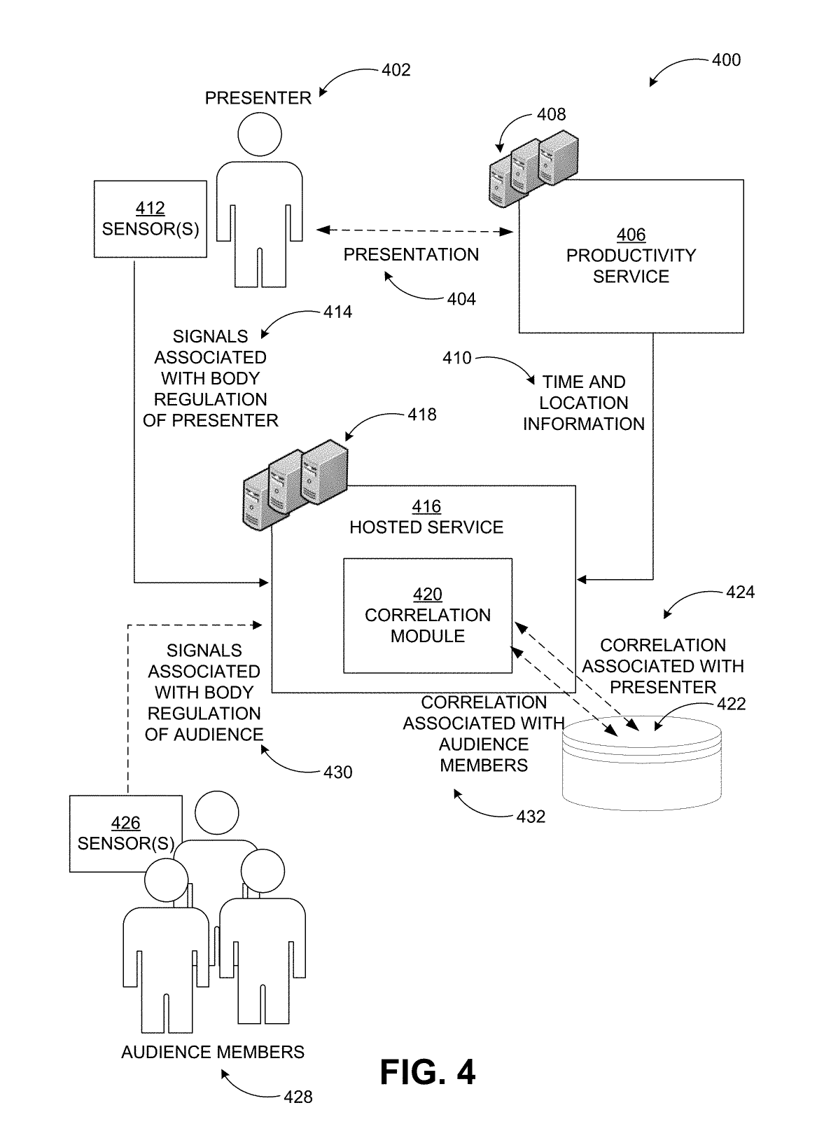

FIG. 4 includes a display diagram illustrating an example scheme to provide stress feedback for a presentation.

As shown in a diagram 400, a presenter 402 may be giving a presentation 404. One or more processing servers 408 of a productivity service 406 may be configured to execute a presentation 404 through a web or client application to be given by a presenter 402. The web or client application may be a word-processing application, a presentation application, a notebook application, or a spreadsheet application that is capable of displaying a continuous presentation based on individual slides, pages, or other similar segments. As the presentation 404 is progressed by the presenter 403, the productivity service 406 may also be configured to collect time and location information 410. Additionally, one or more sensors 412 may be affixed to a body of the presenter 402. The sensors 412 may be remote sensors or the sensors 412 may be integrated into a wearable computing device, such as a watch or a band. The sensors 412 may be configured to collect signals associated with body regulation of the presenter 414, such as heart rate, body temperature, and galvanic skin response, for example, throughout the presentation 404.

A hosted service 416 may be communicatively coupled to the sensors and the productivity service through a communication interface. One or more processors 418 of the hosted service 416 may be configured to execute a correlation module 420 to provide stress feedback information for the presentation 404. For example, the correlation module 420 may be configured to receive the signals 414 from the sensors 412 and the time and location information 410 from the productivity service 406 throughout the duration of the presentation 404. The correlation module 420 may then be configured to determine a stress level of the presenter 402 based on the received signals 414, correlate the determined stress level of the presenter with the time and location information 410, and provide the correlation 424 for display.

The correlation module 420 may be configured to display the correlation 424 in real-time as the presenter is giving the presentation through a website associated with the hosted service 416, for example. Additionally and/or alternatively, the correlation module 420 may be configured to store the correlation 424 in a database 422 associated with the hosted service 416 such that the correlation 424 may be retrieved at a later time for analysis and/or comparison. For example, after the presentation 404 is completed, the presenter 402 may want to know how well he or she did and how they can improve in the future. Therefore, the correlation 424 may be retrieved from the database 422 to show the presenter 402 at which slides, pages, and/or segments of the presentation 404 that the presenter 402 was the most stressed based on the signals received when le presenter 104 was at that particular location in the presentation, example, the correlation 424 may indicate that the presenter 402 was most nervous at slide five of the presentation given elevated heart rate, increased body temperature, and/or increased galvanic skin response while the presenter 402 was discussing information on slide five. Additionally, the presenter 402 may give the presentation 404 on multiple occasions. A correlation may be provided on each occasion and stored in the database. The presenter may then retrieve each of the correlations and compare them in order to see if the presenter has improved.

In some embodiments, one or more additional sensors 426 may be affixed to bodies of one or more audience members 428. The additional sensors 426 may be configured to collect signals associated with body regulation of the audience members 430 throughout the presentation 404. The hosted service 416 may be communicatively coupled to the additional sensors 426 through the communication interface, and the correlation module 420 may be configured to receive the signals 430 from the additional sensors 426. The correlation module 420 may then be configured to determine a stress level of the audience members based on the received signals 430, correlate the determined stress level of the audience members with the time and location information 410, and provide the correlation associated with the audience members 432 for display. In some examples, the correlation associated with the presenter 424 and the correlation associated with the one or more audience members 432 may be aggregated for display.

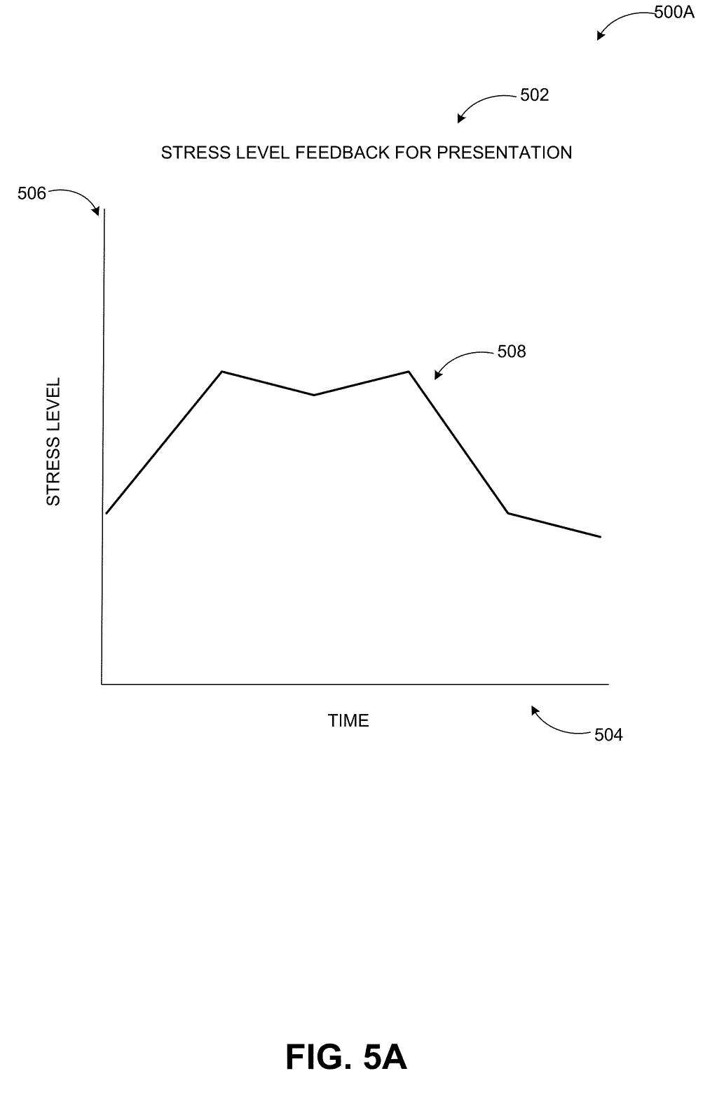

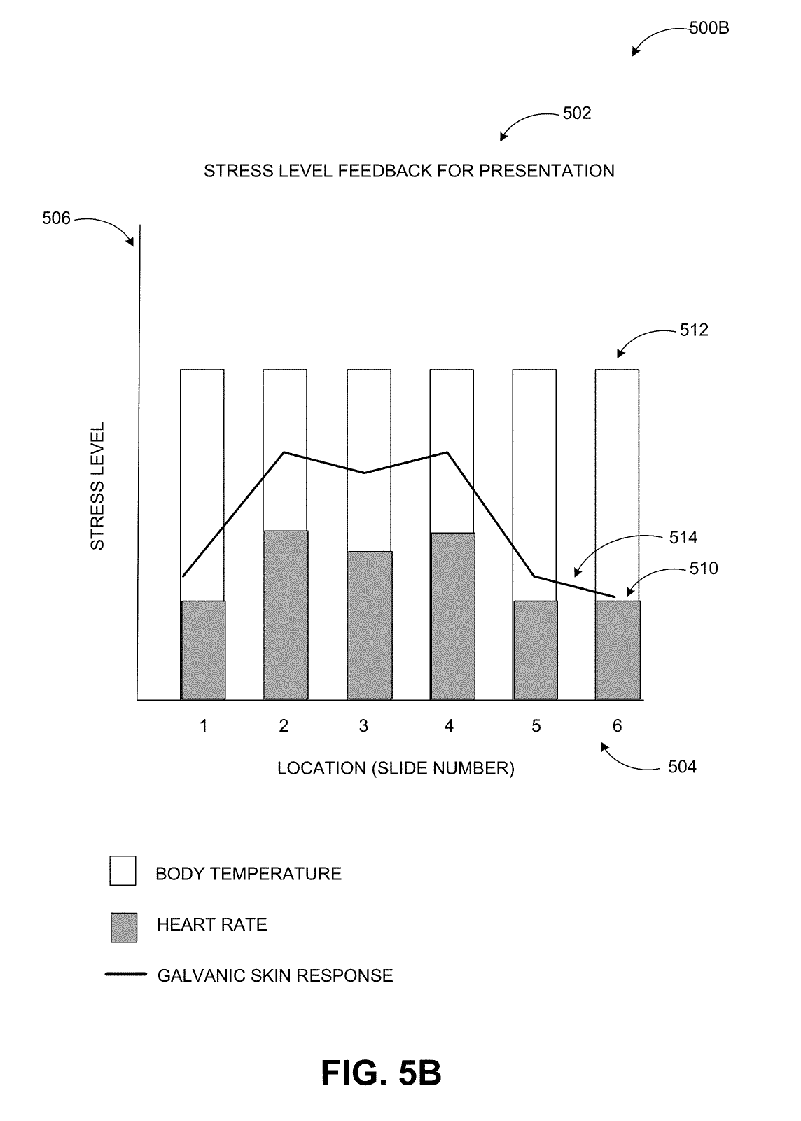

FIGS. 5A-B include display diagrams illustrating example correlations provided for display. As previously discussed, a hosted service may be configured to receive signals associated with body regulation of a presenter throughout the presentation from one or more sensors affixed to a body of the presenter, and time and/or location information as the presentation is progressed by the presenter from a productivity service configured to execute the presentation. The hosted service may then be configured to determine a stress level of the presenter based on the received signals, correlate the determined stress level of the presenter with the time and/or location information, and provide the correlation for display. The correlation may be displayed as a graph 502, wherein an x-axis 504 of the graph represents the time and/or location information and a y-axis 506 represents the determined stress level as shown in diagrams 500A and 500B, One or more of a textual scheme, a graphical scheme, a formatting scheme, an animation scheme, a coloring scheme, a highlighting scheme, and/or a shading scheme may be employed to display the graph 502.

In some embodiments, the determined stress level may be displayed as one aggregate value 508 throughout the duration of the presentation in the graph 502, as shown in the diagram 500A. In other embodiments, a value for each type of the signals collected by sensors is displayed in the graph 502 to represent the determined stress level throughout the duration of the presentation, as shown in the diagram 500B. For example, if the signals collected by the sensors include heart rate, body temperature and galvanic skin response, the graph 502 may display values for heart rate 510, body temperature 512, and galvanic skin response 514 correlating to specific time and/or location information of the presentation when those signals were collected. As illustrated, the presenter may be enabled to analyze the graph 502 to determine that he or she wars most nervous or stressed while they were presenting slide two and four of the presentation based on the elevated heart rate 510, body temperature 512, and galvanic skin response 514 collected by the sensors as slides two and 4 were being presented. The presenter may then use this information to improve the presentation the next time it is given.

The examples provided in FIGS. 1 through 5B are illustrated with specific systems, services, applications, modules, and displays. Embodiments are not limited to environments according to these examples. Provision of stress feedback for presentation may be implemented in environments employing fewer or additional systems, services, applications, modules, and displays. Furthermore, the example systems, services, applications, modules, and notifications shown in FIG. 1 through 5B may be implemented in a similar manner with other values using the principles described herein.

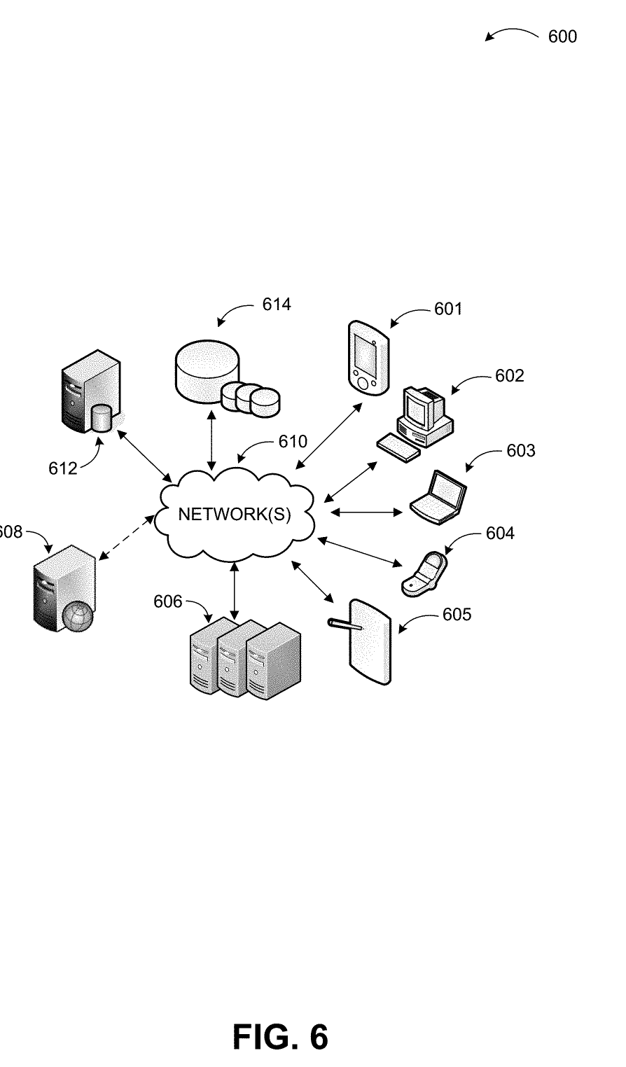

FIG. 6 is a networked environment, where a system according to embodiments may be implemented. In addition to locally installed applications (for example, application 106), a correlation module may also be employed in conjunction with hosted applications and services (for example, a hosted service 120 or a productivity service 318) that may be implemented via software executed over one or more servers 606 or individual server 608, as illustrated in diagram 600. A hosted service or application may communicate with client applications on individual computing devices such as a handheld computer 601, a desktop computer 602, a laptop computer 603, a smart Phone 604, a tablet computer (or slate), 605 (‘client devices’) through network(s) 610 and control a user interface presented to users.

Client devices 601-605 are used to access the functionality provided by the hosted service or application. One or more of the servers 606 or server 608 may be used to provide a variety of services as discussed above. Relevant data may be stored in one or more data stores (e.g. data store 614), which may be managed by any one of the servers 606 or by database server 612.

Network(s) 610 may comprise any topology of servers, clients, Internet service providers, and communication media. A system according to embodiments may have a static or dynamic topology. Network(s) 610 may include a secure network such as an enterprise network, tiara insecure network such as a wireless open network, or the Internet. Network(s) 610 may also coordinate communication over other networks such as PSTN or cellular networks, Network(s) 610 provides communication between the nodes described herein. By way of example, and not limitation, network(s) 610 may include wireless media such as acoustic, RF, infrared and other wireless media.

Many other configurations of computing devices, applications, engines, data sources, and data distribution systems may be employed to provide stress feedback for presentation. Furthermore, the networked environments discussed in FIG. 6 are for illustration purposes only. Embodiments are not limited to the example applications, engines, or processes.

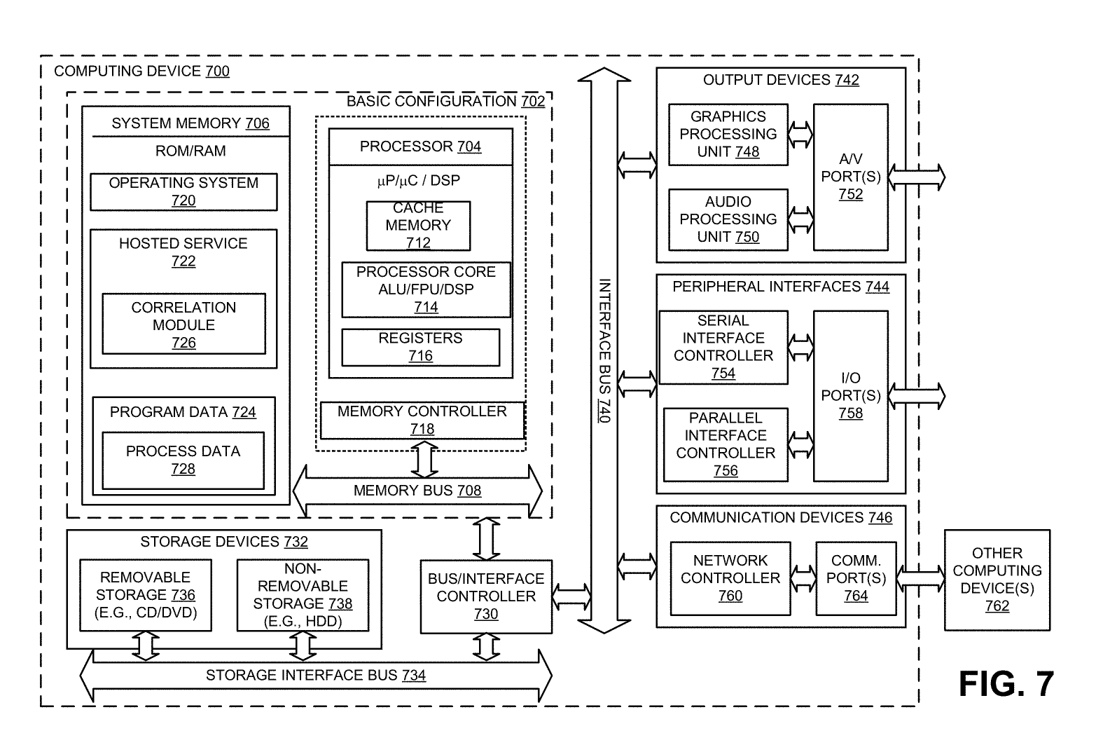

FIG. 7 is a block diagram of an example general purpose computing device, which may be used to provide stress feedback for presentation.

For example, computing device 700 may be used as a server, desktop computer, portable computer, smart phone, special purpose computer, or similar device. In an example basic configuration 702, the computing device 700 may include one or more processors 704 and a system memory 706. A memory bus 708 may be used for communicating between the processor 704 and the system memory 706. The basic configuration 702 is illustrated in FIG. 7 by those components within the inner dashed line.

Depending on the desired configuration, the processor 704 may be of any type, including but not limited to a microprocessor (μP), a microcontroller (μC), a digital signal processor (DSP), or any combination thereof. The processor 704 may include one more levels of caching, such as a level cache memory 712, one or more processor cores 714, and registers 716. The example processor cores 714 may (each) include an arithmetic logic unit (ALU), a floating point unit (FPU), a digital signal processing core (DSP Core), or any combination thereof. An example memory controller 718 may also be used with the processor 704, or in some implementations the memory controller 718 may be an internal part of the processor 704.

Depending on the desired configuration, the system memory 706 may be of any type including but not limited to volatile memory (such as RAM), non-volatile memory (such as ROM, flash memory, etc.) or any combination thereof. The system memory 706 may include an operating system 720, a hosted service 722, and program data 724. The hosted service 722 may include a correlation module 726, which may be an integrated module of the hosted service 722, The correlation module 726 may be, configured to receive signals associated with body regulation of the presenter collected by the one or more sensors throughout the presentation, receive time information collected by the productivity service as the presentation is progressed by the presenter, determine a stress level of the presenter based on the received signals, correlate the determined stress level of the presenter with the time information, and provide the correlation associated with the presenter for display. The program data 724 may include, among other data, process data 728, such as the received signals, the received time and location information, and the correlation, as described herein.

The computing device 700 may have additional features or functionality, and additional interfaces to facilitate communications between the basic configuration 702 and any desired devices and interfaces. For example, a bus/interface controller 730 may be used to facilitate communications between the basic configuration 702 and one or more data storage devices 732 via a storage interface bus 734. The data storage devices 732 may be one or more removable storage devices 736, one or more non-removable storage devices 738, or a combination thereof. Examples of the removable storage and the non-removable storage devices include magnetic disk devices such as flexible disk drives and hard-disk drives (HDDs), optical disk drives such as compact disk (CD) drives or digital versatile disk (DVD) drives, solid state drives (SSD), and tape drives to name a few. Example computer storage media may include volatile and nonvolatile, removable and non-removable media implemented in any method or technology for storage of information, such as computer readable instructions, data structures, program modules, or other data.

The system memory 706, the removable storage devices 736 and the non-removable storage devices 738 are examples of computer storage media. Computer storage media includes, but is not, limited to, RAM, ROM, EEPROM, flash memory or other memory technology, CD-ROM, digital versatile disks (DVDs), solid state drives, or other optical storage, magnetic cassettes, magnetic tape, magnetic disk storage or other magnetic storage devices, or any other medium which may be used to store the desired information and which may be accessed by the computing device 700. Any such computer storage media may be part of the computing device 700.

The computing device 700 may also include an interface bus 740 for facilitating communication from various interface devices (for example, one or more output devices 742, one or more peripheral interfaces 744, and one or more communication devices 746) to the basic configuration 702 via the bus/interface controller 730. Some of the example output devices 742 include a graphics processing unit 748 and an audio processing unit 750, which may be configured to communicate to various external devices such as a display or speakers via one or more A/V ports 752. One or more example peripheral interfaces 744 may include a serial interface controller 754 or a parallel interface controller 756, which may be configured to communicate with external devices such as input devices (for example, keyboard, mouse, pen, voice input device, touch input device, etc.) or other peripheral devices (for example, printer, scanner, etc.) via one or more I/O ports 758. An example communication, device 746 includes a network controller 760, which may be arranged to facilitate communications with one or more other computing devices 762 over a network communication link via one or more communication ports 764. The one or more other computing devices 762 may include servers, computing devices, and comparable devices.

The network communication link may be one example of a communication media, Communication media may typically be embodied by computer readable instructions, data structures, program modules, or other data in a modulated data signal, such as a carrier wave or other transport mechanism, and may include any information delivery media. A “modulated data signal” may be a signal that has one or more of its characteristics sot or changed in such a manner as to encode information in the signal. By way of example, and not limitation, communication media may include wired media such as a wired network or direct-wired connection, and wireless media such as acoustic, radio frequency (RF), microwave, infrared (IR) and other wireless media. The term computer readable media as used herein may include both storage media and communication media.

The computing device 700 may be implemented as a part of a general purpose or specialized server, mainframe, or similar computer that includes any of the above functions. The computing device 700 may also be implemented as a personal computer including both laptop computer and non-laptop computer configurations.

Example embodiments may also include methods to provide stress feedback for a presentation. These methods can be implemented in any number of ways, including the structures described herein. One such way may be by machine operations, of devices of the type described in the present disclosure. Another optional way may be for one or more of the individual operations of the methods to be performed in conjunction with one or more human operators performing some of the operations while other operations may be performed by machines. These human operators need not be collocated with each other, but each can be only with a machine that performs a portion of the program. In other embodiments, the human interaction can be automated such as by pre selected criteria that may be machine automated.

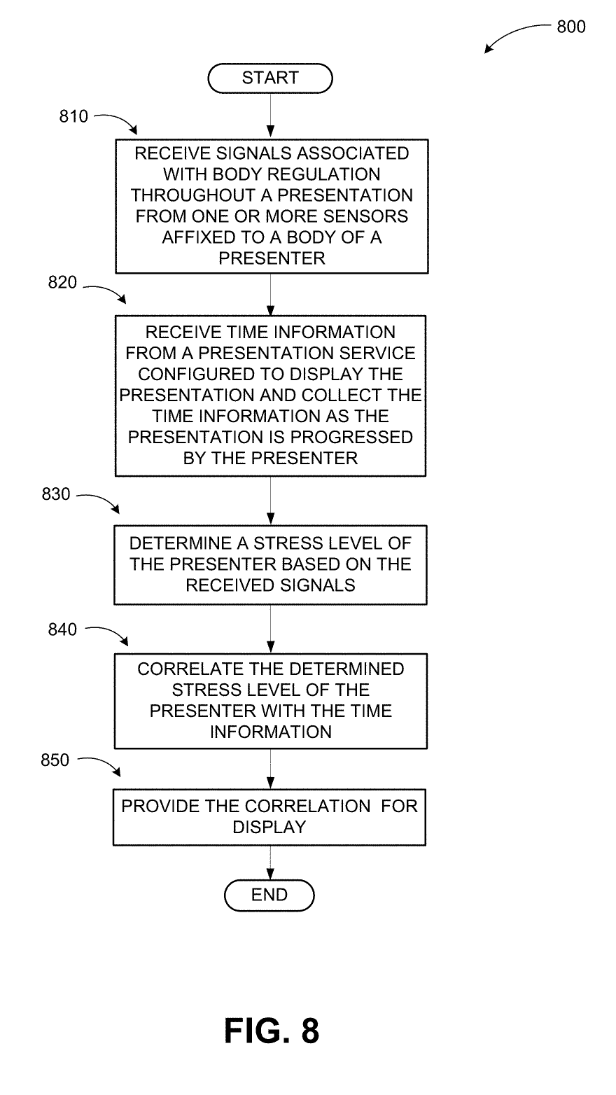

FIG. 8 illustrates a logic flow diagram of a method to provide stress feedback for a presentation. Process 800 may be implemented on a computing device, server, or other system. An example server may comprise a communication interface to facilitate communication between one or more sensors affixed to a body of a presenter, a productivity service configured to execute the presentation, and the server. The example server may also comprise a memory to store instructions, and one or more processors coupled to the memory. The processors, in conjunction with the instructions stored on the memory, may be configured to provide stress feedback for a presentation.

Process 800 begins with operation 810, where the processors are configured to receive signals associated with body regulation of the presenter collected throughout the presentation from the sensors. The received signals may include heart rate, body temperature, and galvanic skin response, for example, along with signals associated with any other body regulations indicative of stress and detectable via body contact based sensors.

At operation 820, the processors are configured to receive time information collected from the productivity service as the presentation is progressed by the presenter. In some embodiments, location information collected by productivity service may also be received, where the location information may include a slide number, a page number, a paragraph number, a line number, or any other similar segmentation number.

At operation 830, the processors may be configured to determine a stress level of the presenter based on the received signals, and at operation 840, the processors may be configured to correlate, the determined stress level of the presenter with the time information. If location information is also received, as discussed previously in conjunction with operation 820, then the processors may be configured to correlate the determined stress level of the presenter with the location information.

At operation 850, the processors may be configured to provide the correlation for display. The correlation may be displayed in real time during the presentation and/or may be stored in a database so that it may be retrieved later for analysis. The correlation may be displayed as a graph, where the x-axis represents time and/or location information and the y-axis represents the stress level of the presenter.

The operations included in process 800 are for illustration purposes. Provision of stress feedback for a presentation may be implemented by similar processes with fewer or additional steps, as well as in different order of operations using the principles described herein. The operations described herein may be executed by one or more processors operated on one or more computing devices, one or more processor cores, specialized processing devices, and/or general purpose processors, among other examples.

According to sonic embodiments, means to provide stress feedback for a presentation may be provided. Examples means may include receiving signals associated with body regulation of a presenter throughout the presentation from one or more sensors affixed to a body of the presenter and receiving time information from a productivity service configured to provide the presentation and collecting the time information as the presentation is progressed by the presenter. Example means may also include determining a stress level of the presenter based on the received signals, correlating the determined stress level of the presenter with the time information, and providing the correlation for display.

According to some methods, methods to provide stress feedback for a presentation may be provided. An example method may include receiving signals associated with body regulation of a presenter throughout the presentation from one or more sensors affixed to a body of the presenter and receiving time information from a productivity service configured to provide the presentation and collecting the time information as the presentation is progressed by the presenter. The example method may also include determining a stress level of the presenter based on the received signals, correlating the determined stress level of the presenter with the time information, and providing the correlation for display.

In other examples, location information collected by the productivity service may be received as the presentation is progressed, by the presenter, where the location information may include a slide number, a page number, a paragraph number, a line number, a presentation element, or other segmentation indicator of the presentation. The determined stress level may be further correlated with the location information.

In further examples, correlation may be displayed as a graph, where an x-axis of the graph may represent the time information and a y-axis may represent the determined stress level. The determined stress level may be displayed as one aggregate value in the graph, A value for each type of the signals collected by the one or more sensors may be displayed in the graph to represent the determined stress level. The correlation may be provided for display in real-time.

According to some embodiments, servers configured to provide stress feedback for a presentation may be described. An example server may include a communication in configured to facilitate communication between one or more sensors affixed to a body of a presenter, a productivity service providing the presentation, and the server, a memory configured to store instructions, and one or more processors coupled to the memory, and a database configured to store the correlation associated with the presenter. The processors, in conjunction with the instructions stored in the memory, may be configured to receive signals associated with body regulation of the presenter collected by the one or more sensors throughout the presentation and receive time information collected by the productivity service as the presentation is progressed by the presenter. The processor may be further configured to determine a stress level of the presenter based on the received signals, correlate the determined stress level of the presenter with the time information; and provide the correlation associated with the presenter for display.

In other embodiments, the signals associated with body regulation may include signals associated with the presenter's heart rate, blood pressure, skin temperature, skin hydration level, and/or galvanic skin response. The database may be configured to store One or more other correlations associated with one or more other presenters performing a same presentation, where the presenter and other presenters may be a part of a group, team, or organization. The processors may be configured to analyze the correlation associated, with the presenter and the other correlations associated with the other presenters performing the same presentation to provide feedback to the group, team, or organization.

In further embodiments, the database may be configured to store one or more other correlations associated with a same presentation performed by the presenter, where the processors may be configured to analyze the correlation and the other correlations associated with the same presentation to provide feedback to the presenter.

According to other examples, systems to provide stress feedback for a presentation. An example system includes one or more sensors affixed to a body of a presenter, where the sensors may be configured to collect signals associated with body regulation, a first server hosting a productivity service, where the productivity service may be configured to provide the presentation and collect time information as the presentation is progressed by the presenter, and a second server. The second server may include a communication interface configured to facilitate communication between the sensor, the first server, and the second server, a memory configured to store instructions, and one or more processors coupled to the memory. The processors, in conjunction with the instructions stored in the memory, may be configured to receive the signals collected by the sensors and the time information collected by the productivity service, determine a stress level of the presenter based on the received signals, correlate the determined stress level of the presenter with the time information, and provide the correlation associated with the presenter for display.

In other examples, sensors include remote sensors and sensors integrated into a wearable, computing device. The presentation may be executed through a web application or a client application associated with the productivity service. The web application or the client application may be a word-processing application, a presentation application, a notebook application, or a spreadsheet, application that is capable of displaying a continuous presentation based on individual slides, pages, paragraphs, lines, or other similar segments.

In further examples, one or more additional sensors may be affixed to bodies of one or more audience members, where the additional sensors may be configured to collect signals associated with body regulation of the audience members. The processors may be further configured to receive the signals associated with body regulation of the audience members, determine a stress level of the audience members based on the received signals, correlate the determined stress level of the one or more audience members with the time information, and provide the correlation associated with the one or more audience members for display. The processors may be further configured to aggregate the correlation associated with the presenter the correlation associated with the one or more audience members for display.

The above specification, examples and data provide a complete description of the manufacture and use of the composition of the embodiments. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims and embodiments.

What is claimed is:

1. A method to provide stress feedback far a presentation, the method comprising:
receiving signals associated with body regulation of a presenter throughout the presentation from one or more sensors affixed to a body of the presenter;
receiving time information from a productivity service configured to provide the presentation and collecting the time information as the presentation is progressed by the presenter;
determining a stress level of the presenter based on the received signals;
correlating the determined stress level of the presenter with the time information; and
providing the correlation for display.

2. The method of claim 1, further comprising:
receiving location information collected by the productivity service as the presentation is progressed by the presenter, wherein the location information includes a slide number, a page number, a paragraph number, a line number, a presentation element, or other segmentation indicator of the presentation.

3. The method of claim 2, wherein correlating the determined stress level with the time information comprises:
further correlating the determined stress level with the location information.

4. The method of claim 1, wherein providing the correlation for display comprises:
displaying the correlation as a graph, wherein an x-axis of the graph represents the time information and a y-axis represents the determined stress level.

5. The method of claim 4, wherein the determined stress level is displayed as one aggregate value in the graph.

6. The method of claim 4, wherein a value for each type of the signals collected by the one or more sensors is displayed in the graph to represent the determined stress level.

7. The method of claim 1, wherein providing the correlation for display comprises:
providing the correlation for display in real-time.

8. A server configured to provide stress feedback for a presentation, the server comprising:
a communication interface configured to facilitate communication between one or more sensors affixed to a body of a presenter, a productivity service providing the presentation, and the server;
a memory configured to store instructions;
one or more processors coupled to the memory, wherein the one or more processors, in conjunction with the instructions stored in the memory, are configured to:
receive signals associated with body regulation of the presenter collected by the one or more sensors throughout the presentation;
receive time information collected by the productivity service as the presentation is progressed by the presenter;
determine a stress level of the presenter based on the received signals;
correlate the determined stress level of the presenter with the time information; and
provide the correlation associated with the presenter for display; and
a database configured to store the correlation associated with the presenter,

9. The server of claim 8, wherein the signals associated with body regulation include signals associated with one or more of the presenter's heart rate, blood pressure, skin temperature, skin hydration level, and galvanic skin response.

10. The server of claim 8, wherein the database is configured to, store one or more other correlations associated with one or more other presenters performing a same presentation, wherein the presenter and one or more other presenters are a part of a group, team, or organization.

11. The server of claim 10, wherein the one or more processors are configured to analyze the correlation associated with the presenter and the one or more other correlations associated with the one or more other presenters performing the same presentation to provide feedback to the group, team, or organization.

12. The server of claim 8, wherein the database is configured to store one or more other correlations associated with a same presentation performed by the presenter.

13. The server of claim 12, wherein the one or more processors are configured to analyze the correlation and the one or more other correlations associated with the same presentation to provide feedback to the presenter.

14. A system to provide stress feedback for a presentation, the system comprising:
one or more sensors affixed to a body of a presenter, wherein the one or more sensors are configured to collect signals associated with body regulation;
a first server hosting a productivity service, wherein the productivity service is configured to provide the presentation and collect time information as the presentation is progressed by the presenter; and
a second server comprising:
a communication interface configured to facilitate communication between the sensor, the first server, and the second server;
a memory configured to store instructions; and
one or more processors coupled to the memory, wherein the one or more processors, in conjunction with the instructions stored in the memory, are configured to:
receive the signals collected by the one or more sensors and the time information collected by the productivity service;
determine a stress level of the presenter based on the received signals;
correlate the determined stress level of the presenter with the time information; and
provide the correlation associated with the presenter for display.

15. The system of claim 14, wherein the one or more sensors include remote sensors and sensors integrated into a wearable computing device.

16. The system of claim 14, wherein the presentation is executed through a web application or a client application associated with the productivity service.

17. The system of claim 16, wherein the web application or the client application is a word-processing application, a presentation application, a notebook application, or a spreadsheet application that is capable of displaying a continuous presentation based on individual slides, pages, paragraphs, lines, or other similar segments.

18. The system of claim 14, further comprising:
one or more additional sensors affixed to bodies of one or more audience members, wherein the one or more additional sensors are configured to collect signals associated with body regulation of the one or more audience members.

19. The system of claim 18, wherein the one or more processors are further configured to:
receive the signals associated with body regulation of the one or more audience members;
determine a stress level of the one or more audience members based on the received signals;
correlate the determined stress level of the one or more audience members with the time information; and
provide the correlation associated with the one or more audience members for display.

20. The system of claim 19, wherein the one or more processors are further configured to:
aggregate the correlation associated with the presenter and the correlation associated with the one or more audience members for display.
